(19) United States
(12) Patent Application Publication
(10) Pub. No.: US 2020/0131459 A1
(43) Pub. Date: Apr. 30, 2020

(54) MICROPLATE COVERS FOR ENVIRONMENTAL CONTROL AND AUTOMATION

(71) Applicant: FenoLogica Biosciences, Inc., Seattle, WA (US)

(72) Inventors: Michael Baum, Bellevue, WA (US); Sean MacLeod, Seattle, WA (US)

(21) Appl. No.: 16/630,373

(22) PCT Filed: Jul. 9, 2018
(86) PCT No.: PCT/US2018/041342

§ 371 Date: Jan. 10, 2020

Related U.S. Application Data
(60) Provisional application No. 62/530,724, filed on Jul. 10, 2017.

(51) Int. Cl. C12M 1/32; C12M 1/00; C12M 1/34

(52) U.S. Cl. CPC C12M 23/12; C12M 41/00; C12M 31/00

(57) ABSTRACT

Disclosed herein are microplate covers that seal microplates and the wells within microplates to retain or control desired environmental conditions within the test environment, such as moisture level, oxygen content, etc. In some embodiments, the visibility and light transmission, refractive, and reflective properties through and from the tray covers can be controlled to facilitate sensing of tray contents via optical or other means.

### CROSS-REFERENCE

This application claims the benefit of U.S. Provisional Patent Application No. 62/530,724, filed Jul. 10, 2017, the entire disclosure of which is incorporated by reference.

### TECHNICAL FIELD

This disclosure is generally related to the detection and analysis of phenotypes. More particularly, this disclosure is related to the automated detection, analysis, and classification of phenotypes of cellular assays.

### BACKGROUND

Microplates are trays with containment walls commonly used in analytical research and diagnostic testing applications. Microplates come in many different sizes and shapes. Manufacturers frequently customize the microplate materials, geometries, and tolerances to accommodate the needs of specific applications and equipment. For example, the layout of dividing walls and wells within the microplates varies greatly depending on application.

Microplates are sometimes sealed to prevent contamination and for other reasons. The variety of microplates available has led to the use of manually applied tape and film type wraps to seal microplates.

### SUMMARY OF THE INVENTION

Disclosed herein are microplate covers that seal microplates and the wells within microplates during assays (e.g., to prevent contamination, facilitate handling and sample analysis, and to retain or control desired environmental conditions within the test environment, such as moisture level, oxygen content (or mixed gas), pressure, etc). In some embodiments, the visibility and light transmission, refractive, and reflective properties through and from the tray covers can be controlled to facilitate sensing of tray contents via optical or other means. In some embodiments, other types of sensors are imbedded in the cover to monitor temperature, pressure, gas content or other environmental conditions of interest. In some embodiments, membranes or channels can be embedded into a microplate cover to facilitate control aspects of the controlled environment within a joined microplate and microplate cover, such as sample manipulation (e.g., introduction, removal, or modification with reagents), gas content, vapor pressure and content, liquid or pressure within each plate or well.

The variety of microplate cover applications, combined with the wide variety of commercially-available microplate geometries described above, present a challenge when integrating microplates with automated instrumentation systems while also introducing a wide set of environmental conditions for highly multiplexed analysis of a small or large number of samples. Disclosed herein are devices and systems of customizable microplate covers which facilitate repeatable and reliable environmental control and automation of tray handling for sensing, analysis, and intra-experimental sample extraction.

In various aspects, the present disclosure provides methods, kits, and apparatuses for assaying a sample. In various aspects, the present disclosure provides apparatuses comprising an identification feature and a microplate cover comprising an interface configured to form a seal between the microplate cover and a vessel, at least a portion of the microplate cover comprising an optically permissive material. In some aspects, the interface of the microplate cover comprises an adhesive material. In some aspects, the vessel is a culture vessel. In some aspects, the vessel is a microplate. In some aspects, the vessel comprises a solid culture matrix. In some aspects, the solid culture matrix is selected from the group consisting of: agar, agarose, carrageenan, gelatin, phytagel, aliginic acid, and guar gum. In some aspects, the vessel comprises a liquid culture medium.

In some aspects, the microplate cover comprises a gas-impermeable barrier when sealed to the microplate. In some aspects, the microplate cover comprises a gas-permeable barrier when sealed to the microplate.

In some aspects, the microplate cover further comprises an alignment feature. In some aspects, the alignment feature comprises an aperture, an indentation, or a protrusion. In some aspects, the alignment feature is configured to receive an alignment pin. Some apparatuses disclosed herein further comprise an alignment pin. In some aspects, the alignment pin is configured to position at least one microplate cover. In some aspects, the indentation comprises a notch or groove.

In some aspects, the microplate cover further comprises an orientation feature. In some aspects, the orientation feature comprises a bevel, recess, or protrusion on the microplate cover.

In some aspects, the microplate cover further comprises a port. In some aspects, the port is a gas-exchange port, the port is connected to an exhaust, or the port is connected to a source of at least one gas. In some aspects, the port comprises a self-healing or self-sealing material. In some aspects the microplate cover comprises a channel.

In some aspects, at least a portion of the microplate cover comprises an optical filter. In some aspects, at least a portion of the microplate cover comprises a lens. In some aspects, the microplate cover comprises at least one sensor.

In some aspects, the present disclosure provides apparatuses further comprising a computer, wherein the computer comprises a processor and a non-transitory memory with executable instructions stored thereon, the instructions when executed causing the processor to: operate one or more detector, operate an alignment pin to position one or more microplate cover, detect the information from an information feature of the one or more microplate covers, position one or more microplate covers based on the information detected from the information feature, position one or more microplate covers based on the orientation feature of the one or more microplate covers, operate one or more light source, operate a gas management system, operate a liquid handling system, and/or operate a tray handling system.

In various aspects, the present disclosure provides apparatuses for assaying a sample, comprising a microplate cover comprising an optical filter or a lens feature; a computer comprising a processor and a non-transitory memory with executable instructions stored thereon, the instructions when executed causing the processor to: operate one or more detectors, operate an imaging system, operate a light source, operate a liquid handling system, operate a gas management system, and/or operate a tray handling system. In some aspects, the apparatuses disclosed herein further comprise a microplate. In some aspects, the microplate is joined to the microplate cover. In some aspects, the volume bounded by the joined microplate and microplate cover comprises one or more samples. In some aspects, the microplate cover comprises one or more ports, an alignment feature, an orientation feature, and/or an information feature. In some aspects, the microplate cover comprises a plurality of sub-regions. In some aspects, the microplate cover comprises no more than one undivided culture region.

In various aspects, the present disclosure provides kits for assaying a sample, comprising a microplate cover, one or more removable liners, and/or a microplate.

In various aspects, the present disclosure provides methods for assaying a sample, comprising: joining a microplate cover to a microplate, wherein the microplate cover comprises an information feature, detecting information from the information feature, positioning the microplate based on the information from the information feature, collecting a first aliquot of a sample from a first region of the microplate, detecting a second aliquot of the sample from a second region of the microplate, detecting a pressure, pH, humidity level, temperature, or gas content in a volume between the joined microplate cover and microplate, adding a volume of one or more gases, removing a volume of one or more gases, detecting a sample through a lens in the microplate cover, or detecting a sample through an optical filter in the microplate cover. In some aspects, the imaging comprises detecting a sample through a lens in the microplate cover, or detecting a sample through an optical filter in the microplate cover. In some aspects, the collecting, imaging, or detecting is initiated by determining a phenotype of a sample. In some aspects, the collecting, imaging, or detecting is initiated by determining that an environmental condition in one or more volumes of the microplate has exceeded a threshold value. In some aspects, the collecting, imaging, or detecting is initiated at a predetermined time point. In some aspects, the joining comprises forming a gas-impermeable seal between the microplate cover and the microplate. In some aspects, the joining comprises forming a gas-permeable seal between the microplate cover and the microplate. In some aspects, the imaging or detecting is performed for a predetermined duration of time. In some aspects, the detecting comprises imaging at least one aliquot of the sample. In some aspects, the microplate cover comprises a port. In some aspects, the port comprises a gas-exchange port.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a microplate and microplate cover according to some embodiments;

FIG. 1B shows a microplate cover installed on and sealing a microplate according to some embodiments;

FIG. 2 shows a microplate cover according to some embodiments;

FIG. 3A shows a microplate cover with adhesive material and a microplate according to some embodiments.

FIG. 3B shows a microplate cover with adhesive material and a microplate according to some embodiments.

FIG. 4 shows a microplate cover installed on and sealing a petri dish according to some embodiments;

FIG. 5 shows a microplate cover according to some embodiments;

FIG. 6 shows a microplate cover comprising a plurality of channels according to some embodiments.

FIG. 7A shows a microplate cover with a plurality of features and a microplate according to some embodiments.

FIG. 7B shows a microplate cover with a plurality of features and a microplate according to some embodiments.

FIG. 7C shows a microplate cover with a plurality of features and a microplate according to some embodiments.

FIG. 7D shows a microplate cover with a plurality of features and a microplate according to some embodiments.

FIG. 8 shows a microplate cover with markings and a microplate according to some embodiments.

FIG. 9A shows a microplate cover with optical features and a microplate imaging system according to some embodiments;

FIG. 9B shows a microplate cover with optical features and a microplate imaging system according to some embodiments.

FIG. 10A shows an exploded view of a microplate cover assembly according to some embodiments;

FIG. 10B shows an assembled view of the microplate cover assembly of FIG. 10A according to some embodiments;

### DETAILED DESCRIPTION OF THE INVENTION

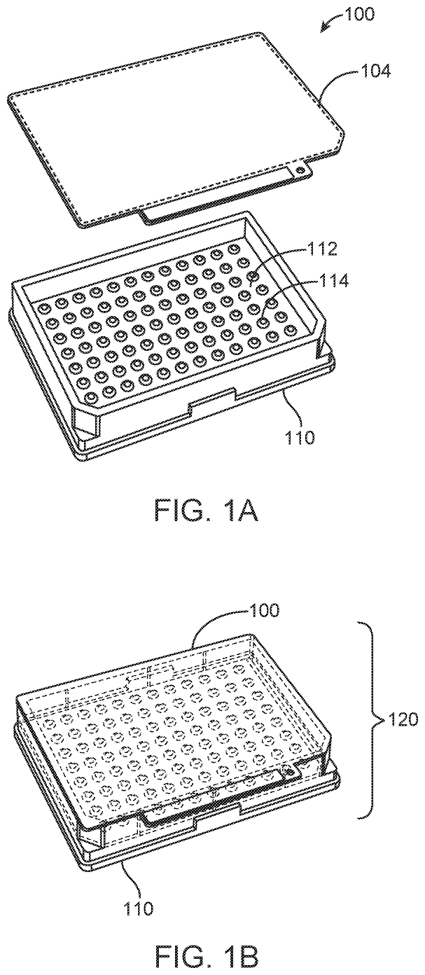

Referring to FIGS. 1A and 1B, a microplate cover 100 and microplate 110 are shown. Microplate cover 100 can join to (e.g., attach to, adhere to, fasten to, or rest upon) the microplate 110. When microplate cover 100 and microplate 110 are joined together (e.g., as shown in FIG. 1B) to form a joined microplate and cover 120, a controllable environment comprising one or more culture region 112 and/or one or more culture sub-region 114 can be formed within the joined microplate and cover 120. For example, one or more culture region 112 and/or one or more culture sub-region 114 of a controlled environment formed by joining microplate cover 100 and microplate 110 can be entirely encompassed by joined microplate and cover 120.

Microplate cover 100 can be used to cover and/or to seal one or more samples within the controllable environment for experimentation. In some embodiments, one or more biological assays (e.g., such as a biological assay comprising a step for imaging cellular growth or cell colony growth, a colorimetric or fluorescence reporter assay, a gene expression assay, a DNA copy number variation assay, an agar invasion assay, a substrate adherence assay, a plasmid loss or maintenance assay, a RNA or DNA transcriptomics assay, a cellular differentiation or reprogramming assay, an assay for determining competition and/or mutual benefit between heterogeneous cell types, a drug efficacy or drug toxicity assay, a cell viability assay, a cell motility assay, or a cell colony morphology assay such as a bacterial cell colony morphology assay, an immunosorbent assay, an animal cell colony morphology assay, a yeast cell colony morphology assay, a multiplex assay for variant effects, etc.) can be performed on one or more samples. As described herein, optical properties and/or feature(s) of microplate cover 100 can improve the accuracy, precision, or speed of a biological assay or step thereof.

A sample can comprise any cell type in any quantity or mixture, including but not limited to yeast, bacteria, animal cells (e.g., mammalian cells such as human, rat, mouse, pig, or non-human primate cells), plant cells, and molds. In some cases, a sample can comprise a cancer cell or a non-cancerous cell. In some cases, a sample comprises a single cell, such as a single cell inoculated onto or into a culture medium (e.g., a solid culture medium such as agar or a liquid medium, such as cell culture medium) located within joined microplate and cover 120. In some cases, a sample comprises a plurality of cells, such as a colony of cells or a tissue sample inoculated or grown in or on a culture medium. As described herein, various embodiments of microplate covers 100, described herein, can be joined to a wide variety of types and sizes of microplate 110, allowing for greater flexibility and customization of low- and high-throughput biological assay design than existing technologies.

Microplate cover 100 can comprise various materials to enable visual inspection or sensing of the samples within a microplate 110 that is sealed to the microplate cover 100. For example, microplate cover 100 can be constructed of one or more optically permissive materials (e.g., transparent or translucent materials), such as polystyrene, polycarbonate, acrylic, or glass. In some cases, microplate cover 100 can be constructed of a blend of transparent or translucent materials, such as a blend of any one of polystyrene, polycarbonate, acrylic, or glass. In some embodiments, microplate cover 100 can be wholly or partially constructed of opaque or semi-opaque materials, which can enable diffusion of lighting or integration of sensors.

Microplate cover 100 can comprise a plurality of discrete sections wherein at least two discrete sections of the microplate cover comprise materials with different optical properties (e.g., different degrees or extents of transparency, optical filtration, polarization, or refraction). A first discrete section of a microplate cover comprising a plurality of discrete sections (e.g., wherein at least two discrete sections of the microplate cover comprise materials with different optical properties) may be of the same thickness or of a different thickness a second discrete section of the microplate cover. An area of microplate cover 100 comprising transparent, translucent, opaque, or semi-opaque materials may span the area of the microplate cover 100 that is within the border of an adhesive material disposed on a surface (e.g., a surface in contact with microplate 110 when microplate cover 100 is joined with microplate 110). In some cases, an area of microplate cover 100 comprising transparent, translucent, opaque, or semi-opaque materials may be shaped to match the shape of the microplate 110 that the microplate cover 100 is attached to or configured to be attached to. For example, one or more portions of microplate cover 100 comprising transparent, translucent, opaque, or semi-opaque materials may be shaped as a circle, square, rectangle, triangle, star, or other polygon and may be located at a position corresponding to a location of a microplate 110 at which a sample is disposed (e.g., a position directly above the sample when the microplate cover is joined to the microplate or a position substantially above the sample when the microplate cover is joined to the microplate). Anti-reflection, anti-auto-fluorescing, hydrophobic, hydrophilic, light wavelength filtering, biologic or nutrient-containing, or other coatings may be applied to surfaces of the covers or contained within the cover materials themselves. For example, a surface of microplate cover 100 (e.g., a surface of a discrete section of microplate cover 100, such as a feature of microplate cover 100) that will be closest to or above the sample when microplate cover 100 is joined to microplate 110 can be hydrophobic (e.g., by coating or treating with a hydrophobic substance), and such a hydrophobic surface of a microplate cover can reduce condensation and improve optical clarity during imaging assays and steps.

Microplate cover 100 can comprise a plurality of sections comprising materials having different rigidity. In some cases, a portion of microplate cover 100 (e.g., a section of microplate cover 100) can be a rigid material, such as a plastic, a metal, a metal alloy, or a ceramic. A microplate cover 100 comprising one or more rigid portion can have more structural strength (e.g., compressive or tensile strength) than a microplate cover that does not comprise a rigid material. A microplate cover 100 having improved structural strength can be useful for protecting samples from physical deformation and for maintaining pressure conditions within a joined microplate and cover 120. A portion of a microplate cover 100 can comprise a flexible material, such as a rubber, an epoxy, a polymer (e.g., a fluoropolymer), or a thin metal (e.g., a metal foil such as aluminum foil). In some cases, a microplate cover 100 comprising a flexible material can allow indirect control over pressure within a joined microplate 110 and microplate cover 100. For example, by varying pressure external to a joined microplate 110 and microplate cover 100 a flexible material comprising a portion of the microplate cover can deform, inducing pressure changes within the microplate and microplate cover proportional to the variations in pressure external to the joined microplate and microplate cover. In some case, a portion of microplate cover 100 comprising a flexible material (e.g., a self-healing or self-sealing material such as a self-sealing polymer or rubber) can be used to introduce or extract reagents (e.g., inject, inoculate, vent, or remove, one or more sample, gas, liquid, drug, etc.) into or out of the space between a joined microplate and cover 120 (e.g., into or out of one or more culture region or culture sub-region).

Microplate 110 can be an open microplate having no dividers or other form of compartmentalization. In some cases, microplate 110 can comprise a plurality of dividers (e.g., walls). In some cases, a divider of microplate 110 can have a height equal to or less than the height of the external walls of microplate 110 (e.g., full height walls or partial height walls, respectively). One or more divider of microplate 110 can partially or fully define a culture region 112 or culture sub-region along with one or more of a portion of microplate cover 100 or a portion of an interior surface of microplate 110. In some cases, a divider of microplate 110 is a vertical divider, such as a wall of a well (e.g., wherein the vertical divider is perpendicular or substantially perpendicular to the largest surface of microplate 110 or microplate cover 100). In some cases, a divider of microplate 110 is a horizontal divider, such as a permeable or porous membrane insert (e.g., wherein the horizontal divider comprises a surface parallel or substantially parallel to the largest interior surface of microplate 110 or microplate cover 100).

Microplate 110 can comprise any shape or format. For example, microplate 110 can be a standard culture format, such as a 1-well, 4-well, 6-well, 12-well, 24-well, 48-well, 96-well, 384-well or 1536-well microplate. A well of microplate 110 can be a round-bottom (or U-bottom) well, a C-bottom well, a V-bottom well, or a flat-bottom (or F-bottom) well (e.g., as defined by a cross-section of the well parallel to the largest surface of a microplate cover 100 joined to microplate 110). A well of microplate 110 can be any shape as evaluated by a cross-section parallel to the largest surface of a microplate cover 100 joined to microplate 110. For example, a well of microplate 110 can be round, rectangular, square, elliptical, star-shaped, or moon-shaped (e.g., as defined by a cross-section of the well parallel to the largest surface of a microplate cover 100 joined to microplate 110).

Microplate 110 can also be a round vessel of any diameter having a circular, elliptical, or circular cross-section (e.g., as defined by a cross-section of microplate 110 parallel to that of the largest surface of a joined microplate cover 100). For example, microplate 110 can be a petri dish or round culture plate of any diameter. Microplate 110 can also be an open basin of any cross-sectional shape (for example, a culture region of a culture flask or micro-fermentation tube(s)). Microplate 110 can have an external dimension of 1.00 mm to 4.00 mm, 4.00 mm to 6.00 mm, 6.00 mm to 10.00 mm, 10.00 mm to 14.20 mm, 14.20 mm to 25.00 mm, 25.00 mm to 50.00 mm, 50.00 mm to 75.00 mm, 75.00 mm to 85.43 mm, 85.43 mm to 85.48 mm, 85.48 mm to 100.00 mm, 100.00 mm to 115.00 mm, 115.00 mm to 127.71 mm, 127.71 mm to 127.76 mm, 127.76 mm to 135.00 mm, 135.00 mm to 150.00 mm, 150.00 mm to 175.00 mm, 175.00 mm to 200.00 mm, or larger than 200.00 mm. An upper edge or surface of microplate 110 (e.g., an upper edge or surface of a divider of microplate 110) can have a thickness of 0.01 mm to 0.10 mm, 0.10 mm to 0.50 mm, 0.50 mm to 1.00 mm, 1.00 mm to 1.50 mm, 1.50 mm to 2.50 mm, 2.50 mm to 5.00 mm, 5.00 mm to 10.00 mm, or 10.00 mm to 15.00 mm.

A culture region 112 can be an area or volume between an interior surface of microplate cover 100 and microplate 110. Culture region 112 can be divided into a plurality of culture sub-regions 114 (e.g., by one or more dividers of microplate 110). In some cases, a first culture region or sub-region can be distinguished from and/or physically separated from a second culture region or sub-region by a portion of joined microplate and cover 120, wherein a portion of microplate cover 100 is joined to microplate 110 (e.g., by an adhesive material).

Surfaces of microplate 110, such as interior walls and/or internal surfaces of external walls, can comprise boundaries of a culture region 112 and/or a culture sub-region 114. A solid or liquid media for sample growth, culture, or assay interrogation (e.g., experimentation) can be located in a culture region 112 or sub-region 114 (e.g., a culture region or culture sub-region) between an interior surface of microplate cover 100 and microplate 110.

A controlled environment within joined microplate and cover 120 can comprise a plurality of culture areas and/or a plurality of culture sub-regions. For example, joined microplate and cover 120 can comprise 1, 2, 3, 4, 5, 6, 7, 8, 9, 10, 11, 12, 13, 14, 15, 16, 17, 18, 19, 20, 24, 36, 48, 96, 192, 384, 1536, from 12 to 24, 24 to 48, from 48 to 96, from 96 to 192, from 192 to 384, from 384 to 1536, from 1536 to 6, 154, from 6, 154 to 10,000, or more than 10,000 culture regions or 1, 2, 3, 4, 5, 6, 7, 8, 9, 10, 11, 12, 13, 14, 15, 16, 17, 18, 19, 20, 24, 36, 48, 96, 192, 384, 1536, from 12 to 24, 24 to 48, from 48 to 96, from 96 to 192, from 192 to 384, from 384 to 1536, from 1536 to 6, 154, from 6, 154 to 10,000, or more than 10,000 culture sub-regions. Culture regions and culture sub-regions can be two-dimensional regions in contact with a surface of microplate 110 or three-dimensional regions (e.g., volumes) that are at least partially bounded by a portion of microplate 110, or internal walls within microplate 110. A culture region 112 or culture sub-region 114 can defined by one or more of: at least a portion of a wall of microplate 110, at least a portion of the bottom of microplate 110, or at least a portion of an internal wall or divider of microplate 110. For example, culture region 112 or culture sub-region 114 can comprise an area or volume within a well of microplate 110. Microplate cover 100 may wholly or partially seal define one or more culture regions or one or more sub-regions (e.g., microplate wells) within the volume between a joined microplate 110 and the microplate cover.

In some cases, a culture region 112 or sub-region 114 can be defined or informed by a physical edge of a sample. For example, a culture sub-region can be defined by the edge(s) of one or more cells (e.g., the outer edges of a first colony of cells cultured in the same culture region as a second colony of cells). A culture region 112 or culture sub-region 114 can be defined or informed by a physical edge of a substantially two-dimensional sample (e.g., an adherent cell on a culture matrix). In some case, a sample can comprise a substantially three-dimensional object or plurality of objects (e.g., a three-dimensional colony of adherent cells or a cell or colony of cells in suspension culture), and a three-dimensional culture region 112 or culture sub-region 114 can be informed by or defined by the dimensions of the sample. A culture region or culture sub-region can be informed by an edge or dimension of a sample by adding or removing an amount to one or more dimensions of the area or volume of the sample to define one or more dimension of the culture region or sub-region. For example, a culture sub-region can be defined as an area (e.g., a circular area) or volume around an adherent cellular sample that is from 75% to 85%, 85% to 95%, 95% to 99%, 99% to 100%, 100% to 101%, 101% to 105%, 105% to 115%, or 115% to 125% of a diameter of the adherent cellular sample.

In some cases, a culture sub-region can be an area or volume defined by a functionally relevant parameter, such as virtual border defined during an imaging step or volume of or within an area or volume comprising a portion of the joined microplate 110 and microplate cover 100. For example, a culture region 112 or culture sub-region 114 can be defined by a field of view, such as a field of view of an imaging system. In some cases, a culture region 112 or culture sub-region 114 can be defined by the edges of an image detected or recorded by an imaging system or by the volumetric bounds of a series of images detected or recorded by an imaging system, such as a Z-stack of images.

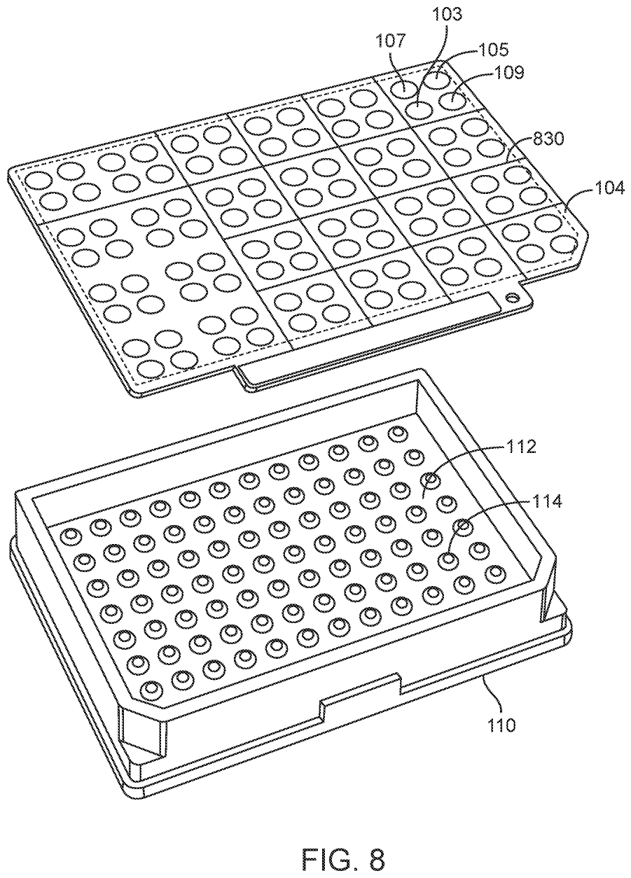

Referring now to FIG. 8, a culture region 112 or sub-region 114 can also be defined by one or more markings 830 on microplate cover 100 or microplate 110, such as colored lines marked on microplate cover 100 or microplate 110 or demarcations engraved or etched into microplate cover 100 or microplate 110. Markings 130 on microplate cover 100 can be any size or shape. For example, marking 830 on microplate cover 100 can comprise one or more circle, triangle, rectangle, square, star shape, moon shape, ellipse, straight line, curved line, letter, number, lattice, etc. Microplate cover 100 and/or microplate 110 can comprise any number of markings 830, which can each define a culture region or culture sub-region. In some cases, a first culture region or first culture sub-region defined by markings 830 can be larger than a second culture region or second culture sub-region defined by markings 830. A first culture region or first culture sub-region defined by markings 830 can be the same size as a second culture region or second culture sub-region defined by markings 830. In some cases, markings 830 on microplate cover 100 can correspond to markings on microplate 110. For example, one or more marking 830 on microplate cover 100 can be the same size and/or shape as one or more marking on microplate 110. One or more marking 830 on microplate cover 100 can be located directly or substantially above one or more marking on microplate 110. In some cases, one or more marking 830 on microplate cover 100 can be smaller than one or more marking on microplate 110 to which it corresponds (e.g., above which it is located). In some cases, one or more marking 830 on microplate cover 100 can be a different shape as one or more marking on microplate 110 to which it corresponds (e.g., above which it is located).

A plurality of culture regions or culture sub-regions can be in any arrangement relative to one another, to microplate 110, or to microplate cover 100. For example, a plurality of culture regions or culture sub-regions, each of which may comprise one or more samples (e.g., one or more cells) can be arranged in a regular array, such as an aligned or staggered array with repeated spacing between samples. In some cases, a plurality of culture regions or culture sub-regions, which may each comprise one or more samples, can be arranged in an irregular array. For example, a plurality of culture regions or culture sub-regions, each comprising one or more cells, can be arranged irregularly (e.g., randomly) on or within a culture media located within joined microplate and cover 120.

In some cases, a culture region or sub-region can comprise 1, 2, 3, 4, 5, 6, 7, 8, 9, 10, 11, 12, 13, 14, 15, 16, 17, 18, 19, 20, from 20 to 30, from 30 to 50, from 50 to 100, from 100 to 200, from 200 to 500, from 500 to 1000, from 1000 to 2000, from 2000 to 5000, from 5000 to 10,000, from 10,000 to 20,000, from 20,000 to 50,000, from 50,000 to 100,000, or more than 100,000 samples. In some cases, an undivided portion of the space between joined microplate cover 100 and microplate 110, which can be a culture region or culture sub-region, can comprise 1, 2, 3, 4, 5, 6, 7, 8, 9, 10, 11, 12, 13, 14, 15, 16, 17, 18, 19, 20, from 20 to 30, from 30 to 50, from 50 to 100, from 100 to 200, from 200 to 500, from 500 to 1000, from 1000 to 2000, from 2000 to 5000, from 5000 to 10,000, from 10,000 to 20,000, from 20,000 to 50,000, from 50,000 to 100,000, or more than 100,000 samples. In some cases, a culture region or culture sub-region can comprise 2, 3, 4, 5, 6, 7, 8, 9, 10, 11, 12, 13, 14, 15, 16, 17, 18, 19, 20, from 20 to 30, from 30 to 50, from 50 to 100, from 100 to 200, from 200 to 500, from 500 to 1000, from 1000 to 2000, from 2000 to 5000, from 5000 to 10,000, from 10,000 to 20,000, from 20,000 to 50,000, from 50,000 to 100,000, or more than 100,000 different samples. For example, a culture region or culture sub-region can comprise 2, 3, 4, 5, 6, 7, 8, 9, 10, 11, 12, 13, 14, 15, 16, 17, 18, 19, 20, from 20 to 30, from 30 to 50, from 50 to 100, from 100 to 200, from 200 to 500, from 500 to 1000, from 1000 to 2000, from 2000 to 5000, from 5000 to 10,000, from 10,000 to 20,000, from 20,000 to 50,000, from 50,000 to 100,000, or more than 100,000 samples (e.g., cells or colonies of cells) having different genetic sequences, different biological sources (e.g., samples isolated from different tissues of the same or different donor organisms), different epigenetic states, or different phenotypes). In some cases, a culture region or culture sub-region can comprise 2, 3, 4, 5, 6, 7, 8, 9, 10, 11, 12, 13, 14, 15, 16, 17, 18, 19, 20, from 20 to 30, from 30 to 50, from 50 to 100, from 100 to 200, from 200 to 500, from 500 to 1000, from 1000 to 2000, from 2000 to 5000, from 5000 to 10,000, from 10,000 to 20,000, from 20,000 to 50,000, from 50,000 to 100,000, or more than 100,000 samples having identical genetic sequences, identical biological sources (e.g., samples isolated from the same tissue of the same or a different donor organisms), identical epigenetic states, or identical phenotypes).

A first aliquot of a sample can have an identical genetic sequence, an identical biological source, an identical epigenetic state, or an identical phenotype as a second aliquot of the sample. In some cases, a first aliquot of a sample can have a different biological source, a different epigenetic state, or a different phenotype as a second aliquot of the sample. In some cases, a first aliquot of a sample can be located in the same culture region as a second aliquot of the sample. In some cases, a first aliquot of a sample can be located in the same culture sub-region as a second aliquot of the sample. In some cases, a first aliquot can be located in a different culture region as a second aliquot of the sample. In some cases, a first aliquot can be located in a different culture sub-region as a second aliquot of the sample.

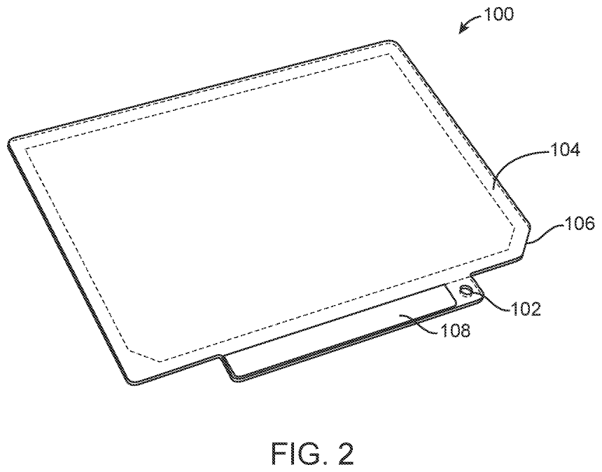

Referring to FIG. 2, microplate cover 100 can include an adhesive material 104 (e.g., an adhesive strip) or mechanical attachment features along the bottom, or microplate facing surface, of the microplate cover 100. In some cases, adhesive material 104 or the mechanical attachment enable rigid attachment of sample containment trays other than microplates (e.g., open format sample containment vessels such as culture dishes, petri dishes, test tubes, custom trays, etc). A portion of a microplate cover comprising an adhesive material 104 can comprise an interface, which can be used to join the microplate cover to a microplate. For example, a contact adhesive or adhesive strip can be disposed on (e.g., applied to or coated on) at least a portion of a surface of microplate cover 100 (e.g., a portion of microplate cover 100 in contact with microplate 110 when the microplate cover and microplate are joined, such as all or a portion of the bottom surface of the microplate cover). The adhesive material 104 may couple the microplate cover 100 to a microplate. In some cases, an adhesive material can be disposed on portions of microplate cover 100 that correspond to portions of microplate 110 that microplate cover 100 will contact when joined with microplate 110. In some cases, an adhesive material may be limited to portions of microplate cover that correspond to portions of microplate 110 that microplate cover 100 will contact when joined with microplate 110. For example, an adhesive material may be disposed (e.g., in a lattice pattern) on a surface of microplate cover 100 where microplate cover 100 will contact the uppermost edges of the outer walls and inner divisions (such as walls of microtiter plate wells) of microplate 110.

Additionally or alternatively, an adhesive material may be disposed on a surface of microplate cover 100 in positions corresponding to other microplate formats. For example, in addition to or alternative to an adhesive material disposed in a lattice pattern on a surface of a microplate cover, an adhesive material may be disposed in a circle 402 on a surface of the microplate cover to facilitate sealing of the microplate cover to a petri dish-format microplate. In some cases, adhesive material may be disposed on a surface of a microplate cover to facilitate sealing the microplate cover to microplates having different dimensions (e.g., external dimensions or dimensions of internal divisions, such as well diameters). For example, an adhesive material may be disposed on the surface of a microplate cover 100 in concentric rings so that the microplate cover can be sealed to a plurality of differently sized petri-dishes or round culture plates.

In some embodiments, the adhesive material may rigidly couple the cover to a microplate such that the assembled microplate cover 100 and microplate 110 may be held in inverted or other positions to help control condensation or suspend biological samples in orientations which may otherwise be inconvenient. In some embodiments, the adhesive bond between the microplate cover 100 may be such that the separation force to separate the microplate 110 from the microplate cover 100 is greater than the gravitational force acting on the microplate 100. Such an embodiment allows the assembled microplate 110 and microplate cover 100 to be lifted by the microplate cover 100. In some embodiments, the adhesive bond may be such that the separation force required to separate microplate 110 from the microplate cover 100 (e.g., during centrifugation, microplate or microplate cover handling, or as a result of internal pressure exerted on the microplate cover or microplate) is greater than the 2, 3, 4, 5, 7, or 10 times the force exerted by gravity on the microplate 110.

In some cases, the seal formed when microplate cover 100 is joined to microplate 110 is a permanent seal. For example, an adhesive material 104 may comprise an adhesive that cross-links or sets permanently on contact, upon exposure to air, upon exposure to certain forms of radiation (e.g., ultraviolet light), or upon mixing with a binary component.

In some embodiments, the adhesive material 104 may be low-tack adhesive to facilitate removal and/or re-use of microplate covers. In some cases, an adhesive material or an adhesive strip can comprise a gas- or liquid-permeable foam gasket.

In some embodiments, the adhesive material 104 may also form a seal between the microplate and the microplate cover 100 to aid in preventing environmental leakage or contamination. In some cases, an adhesive material 104 of microplate cover 100 or a portion of adhesive material 104 of microplate cover 100 can form gas-impermeable or liquid-impermeable barrier when contacted by portion (e.g., a surface, edge, wall, etc.) of microplate 110. In some cases a gas-impermeable or liquid-impermeable barrier formed by contacting adhesive material 104 of microplate cover 100 to a portion of microplate 110 (e.g., one or more structure, such as a surface, edge, or wall) can cause a first culture region 112 or first culture sub-region 114 to be environmentally isolated or substantially isolated from a second culture region or a second culture sub-region of the space between microplate cover 100 and microplate 110 (e.g., wherein the first and second culture regions or culture sub-regions are defined by structures (e.g., surfaces or portions thereof) of microplate cover 100 and/or structures of microplate 110 that are located on opposite sides of the portion (e.g., the structure or structures) joined to microplate cover 100 by adhesive material 104). In some cases, culture regions or culture sub-regions that are environmentally isolated from one another can be prevented from being in fluid communication with one another (e.g., by a portion of microplate cover 100 joined or sealed to a portion of microplate 110 or by a valve of a channel 610 of microplate cover 100). For example, culture regions or sub-regions that are environmentally isolated from one another may be unable to share any or all of the following conditions as a result of being separated by a portion of microplate cover 100 and microplate 110: water vapor, gas content, fluids, solutes, or temperature. In some cases, culture regions or culture sub-regions that are substantially isolated from one another can be prevented from sharing some environmental conditions (e.g., solutes), while allowing other environmental conditions (e.g., fluids such as water) to pass between the substantially isolated regions or sub-regions. In some embodiments the seal may be an air, gas or vapor permeable seal that aids in preventing solid contaminants from entering the microplate wells.

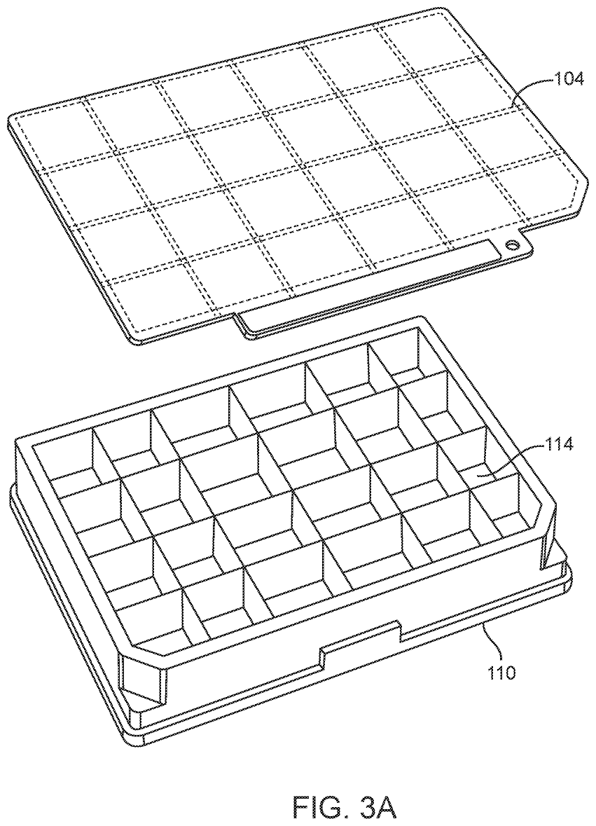

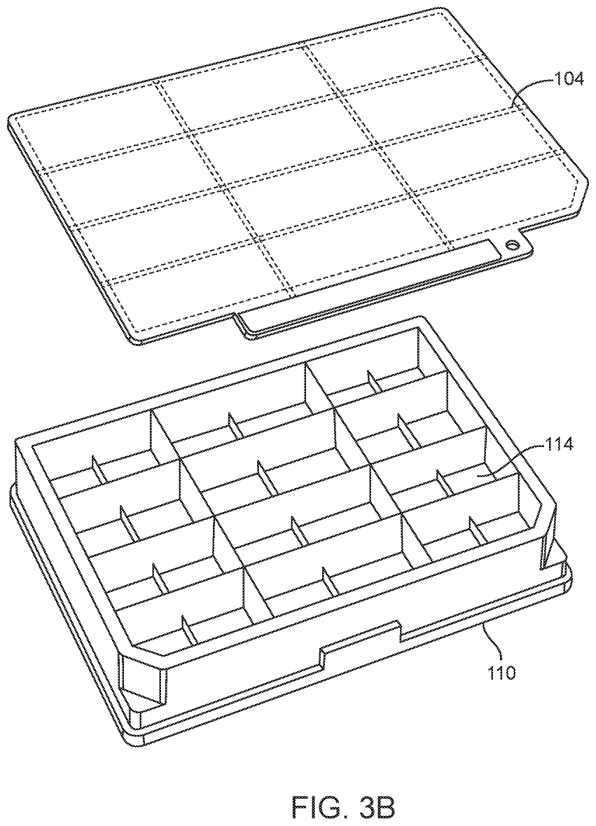

Referring to FIG. 3A, adhesive material 104 may be located at (and, optionally, limited to) portions of microplate cover 100 in contact with one or more portion of microplate 110. In some embodiments, adhesive material 104 is located on (and, optionally, limited to) all portions of microplate cover 100 in contact with any portion of microplate 110. In some embodiments, adhesive material 104 is located on (and, optionally, limited to) portions of microplate cover 100 that can be used to define divisions between culture regions or sub-regions 114 when microplate cover 100 is joined with microplate 110. In some cases, adhesive material 104 located on microplate cover 100 such that all culture regions or culture sub-regions 114 of joined microplate cover 100 and microplate 110 are environmentally isolated from one another by portions of microplate cover 100 and microplate 110 joined by adhesive material 104. For example, a microplate cover 100 in contact with (e.g., joined with) a microplate 110 having 6, 12, 24, 48, 96, 384, or 1536 full-height wells (e.g., wells with upper edges level with one or more external wall of microplate 110) can comprise adhesive material 104 at located on and limited to all portions of microplate cover 100 that contact an external or internal wall of microplate cover 100 when microplate cover 100 is joined to microplate 110. Referring to FIG. 3B, adhesive material 104 can be limited to portions of microplate cover that will be used to define a plurality of culture regions or a plurality of culture sub-regions 114 that are intended to share a culture environment during an assay.

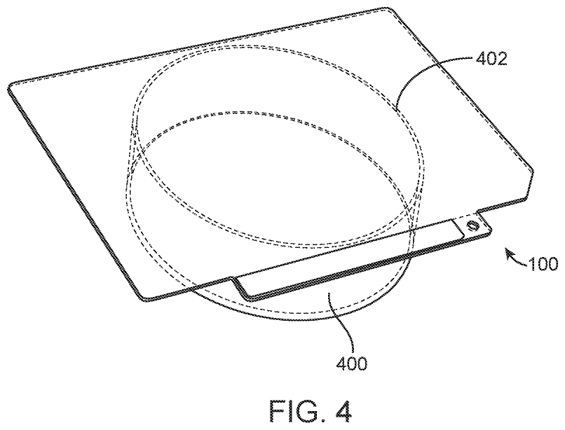

As shown in FIG. 4, the microplate cover 100 may have adhesive material 104, 402 in a shape matching or corresponding to a shape of one or more upper surface or upper edge of a microplate or other sample containment vessel 400, which can be, for example, a petri dish or round culture plate. In some cases, a microplate cover with adhesive material 104, 402 disposed on a surface of the microplate cover in a shape that matches a shape of a sample containment vessel allows for mixed use of various sizes and types of containment trays with a standardized interface on cover plates 100, simplifying use with automated systems or fixturing. For example, an automated system may include sample handling features configured to handle a microplate cover having a particular size and shape. The use of two or more microplate covers having the same size and shape but different coupling configurations (e.g., differently shaped, sized, positioned, or patterned adhesive materials or joining mechanisms) in a system for performing an assay (e.g., an assay comprising an imaging step) can significantly increase the number of different types of sample containers with which the system can interact. For example, a system as described herein can comprise a plurality of different microplate covers configured to be coupled to differently shaped and/or differently sized sample containers, such as microplates and petri dishes of different sizes and configurations (including microplates and culture dishes having different internal wall partitioning). Systems comprising a plurality of microplate covers 100 having compatible handling configuration (e.g., similar external dimensions and/or similar compatible alignment features and/or orientation features) but configured to interact with (e.g., join with or form a seal with) a plurality of differently sized and/or shaped microplates can facilitate higher assay throughput, e.g., by reducing the amount of time needed for system recalibration between differently formatted microplates during performance of assays.

As shown in FIG. 4, the adhesive material 104, 402 on the lower surface of the microplate cover may have a shape that is different than the shape of the perimeter of the microplate cover. As discussed above, an adhesive material can be disposed on microplate cover 100 in patterns including one or more circles, squares, rectangles, or lattices to join the microplate cover to a microplate of any shape, such as a sample containment vessel 400 (e.g., round culture or petri dish). In some cases, the adhesive material is distributed evenly over a surface of microplate cover 100. In some cases, the adhesive material is distributed in a gradient over a surface of a microplate cover.

An adhesive material of microplate 100 can be present on a portion of microplate cover 100 having a dimension (e.g., a width, thickness, or length) of 0.01 mm to 0.10 mm, 0.10 mm to 0.50 mm, 0.50 mm to 1.00 mm, 1.00 mm to 1.50 mm, 1.50 mm to 2.50 mm, 2.50 mm to 5.00 mm, 5.00 mm to 10.00 mm, 10.00 mm to 15.00 mm, 15.00 mm to 25.00 mm, 25.00 mm to 50.00 mm, 50.00 mm to 75.00 mm, 75.00 mm to 85.43 mm, 85.43 mm to 85.48 mm, 85.48 mm to 100.00 mm, 100.00 mm to 115.00 mm, 115.00 mm to 127.71 mm, 127.71 mm to 127.76 mm, 127.76 mm to 135.00 mm, 135.00 mm to 150.00 mm, 150.00 mm to 175.00 mm, 175.00 mm to 200.00 mm, or larger than 200.00 mm. In some cases, adhesive material 104 can be present in a continuous or piecewise continuous straight line or curved line (e.g., a dotted, dashed, or solid line) on a portion of microplate cover 100.

In some embodiments, microplate covers may be attached by mechanical fastening means such as latching “snap” features, such as snap-fits that mate or engage with corresponding features on microplates in addition to or in place of adhesive material. This type of arrangement allows for removal and/or re-use of microplate covers.

As shown in FIG. 2, Microplate cover 100 can comprise one or more features useful in facilitating certain aspects of a biological assay, such as sample identification, microplate identification, automated microplate handling and re-positioning, data collection, and/or data analysis. For example, a microplate cover can comprise one or more of alignment feature 102, orientation feature 106, and information feature 108. In some cases, microplate cover 100 can comprise a plurality of alignment features, orientation features, or information features.

Alignment feature 102 can be useful in facilitating proper joining of microplate cover 100 to microplate 110. In some cases, alignment feature 102 can comprise one or more alignment feature (e.g., a notch or aperture) disposed asymmetrically on an edge of microplate cover 100 such that the one or more alignment features in microplate cover 100 will align with at least one corresponding alignment feature (e.g., at least one notch or aperture of similar size and cross-sectional shape to the alignment feature(s)) of the microplate cover. For example, an alignment feature of microplate cover 100 can be notched on the same rail on which a microplate is notched, and the microplate cover can be precisely guided to contact and join with the microplate. In some cases, an alignment feature of microplate cover 100 comprises an aperture capable of receiving an alignment pin (e.g., of a microplate carrying device, positioning/orientation mechanism (such as a mechanical or robotic handling system), or an imaging system 612 (such as a precision stage used for repeatable positioning during imaging), including those described herein) along which the microplate cover can slide until it contacts and, optionally, joins with microplate 110. In some cases, a microplate cover 100 or joined microplate and cover 120 comprising an alignment feature can be manually aligned or manually positioned (e.g., using the alignment feature and, optionally, an alignment pin). In some cases, a microplate cover 100 or joined microplate and cover 120 comprising an alignment feature can be automatically aligned or automatically positioned (e.g., using the alignment feature and, optionally, an alignment pin controlled by a computer and/or a microplate or tray handling system, which can also be controlled by the computer).

One or more corners of microplate cover 100 may be include orientation feature 106, such as a recessed or notched corner to aid in ensuring proper orientation or “clocking” of the cover and attached microplate. In some cases, such as cases in which a microplate 110 is rotationally symmetrical (e.g., a petri dish or round culture plate), orientation feature 106 can be especially useful in determining the rotational orientation and/or re-orienting the microplate so that one or more steps in an assay (e.g., an assay carried out by an automated system) may be performed with spatial accuracy and precision. For example, orientation feature 106 can be useful in determining a location of a sample or a specific portion of a culture region or sub-region and for reducing variability in analysis of region(s) of interest (such as sample locations or specific portions of a culture region or sub-region), especially when the region(s) of interest is/are not regularly distributed in a culture region or sub-region. In many cases, an orientation feature 106 comprises an asymmetrical feature of microplate cover 100. Orientation feature 106 can comprise an indentation in microplate cover 100 (such as a notch), a protrusion from microplate cover 100 (such as an overhanging tab), or an edge feature of microplate cover (such as one, two, or three beveled or rounded corner(s) on a four-cornered microplate). In some cases, orientation feature 106 comprises one or more visual markings, such as a pigment-based, etched, engraved, or frosted region of microplate cover 100, useful in determining the microplate's orientation. For example, lattice or cross-hairs patterns may be used to aid in determining the orientation of a microplate in an apparatus, as described herein, or in re-orienting (e.g., re-positioning) a microplate in an apparatus, as described herein. In some cases, an orientation feature may be disposed on a surface of microplate cover 100 such that the orientation feature (e.g., a black plus-sign marked on a surface of the microplate cover) is captured during data collection (e.g., during an imaging step) so that orientation determination or spatial re-orientation of collected data may be performed during data processing or analysis. In many cases, an orientation feature can be detected by a computer of an apparatus, as described herein, and can be used to instruct automated or manual re-positioning (e.g., re-orientation) of the microplate 110.

In some cases, a microplate cover 100 or joined microplate and cover 120 comprising an orientation feature can be manually oriented or manually positioned using the orientation feature. In some cases, a microplate cover 100 or joined microplate and cover 120 comprising an orientation feature can be automatically oriented or automatically positioned using the orientation feature.

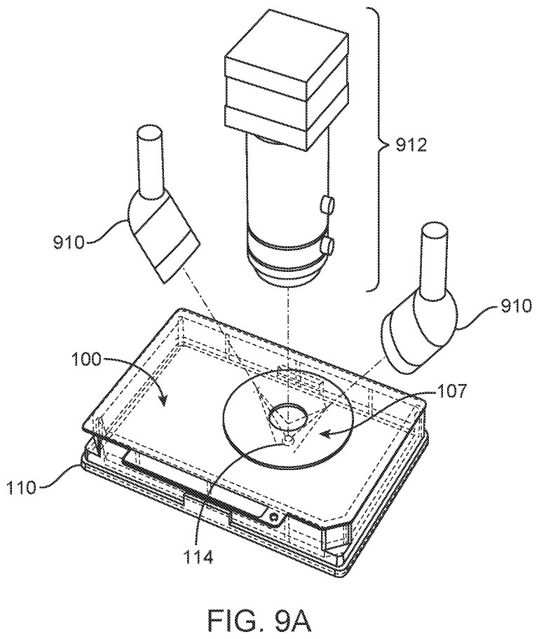

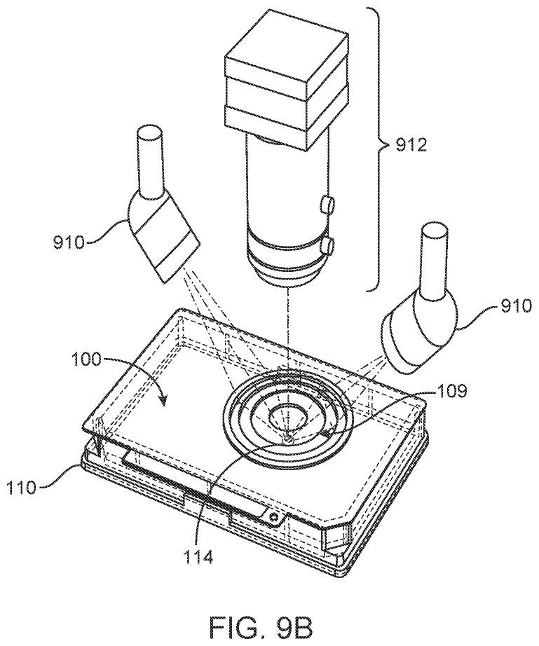

Information feature 108, which can comprise a tab, may extend outside of the microplate footprint (i.e., beyond the outer perimeter of the microplate sidewalls), to provide an area for labelling or identification of the microplate 110 by users or automated instrumentation. In some cases, information feature 108 does not obscure an imaging area (e.g., a culture region or culture sub-region) of microplate 110 during an assay. Information feature 108 may also be used as an alignment feature and/or an orientation feature 106 for users, fixtures, or automated equipment to situate the tray assembly in a repeatable position for transportation, measurement and analysis. The holding or orientation feature 106 may be an aperture or notch though the microplate cover 100 that may receive an alignment pin of a microplate carrying device or imaging system, such as the imaging systems shown in FIGS. 9A and 9B.

Information feature 108 may provide fiducial markings or features in the cover plate may serve as identification, dimensional, orientation, and/or calibration references for measurements taken of samples in the microplate. Information feature 108 can comprise a UPC barcode, a QR code, or a radio frequency identification (RFID) identifier. In some cases, information feature 108 of microplate cover 100 comprises information regarding experimental conditions relevant to the microplate to which the microplate cover is joined, such as experimental conditions, sample identification, timestamping (e.g., for time points or durations of one or more step in a biological assay), treatment groups, standards, and experimental control groups. Information contained or represented by information feature 108 may also comprise instructions that can be detected by an experimental platform in which the microplate cover 100 and microplate 110 are interrogated, manipulated, or used in a biological assay. For example, an information feature (or optionally, an alignment feature and/or orientation feature) can be used to position a joined microplate and microplate cover during intake by an experimental system (e.g., a system which comprises an imaging system 612, as in FIGS. 9A and 9B) and then read by a detector to instruct the experimental system which experimental conditions, reagents, time points, etc. should be employed during an assay. In some cases, information feature 108 can be configured to be modified during an assay. For example, an experimental system (e.g., a system which comprises an imaging system 912, as in FIGS. 9A and 9B) can append, delete from, or otherwise modify information feature 108 in a way that can be detected and interpreted (and, optionally, implemented) by subsequent systems in a series of connected (and, optionally, automated) experimental systems (e.g., a plate reader, an incubator, a microplate handling system, or an imaging system). Modification to an information system can comprise rewriting an RFID information (e.g., information on a 13.56 Mhz or 860-960 Mhz RFID tag), printing or etching (e.g., laser etching) information on the information feature (such as words, a barcode, or tally marks), or physically modifying the information feature (e.g., scoring the material of the microplate cover, or punching one or more hole through the information feature).

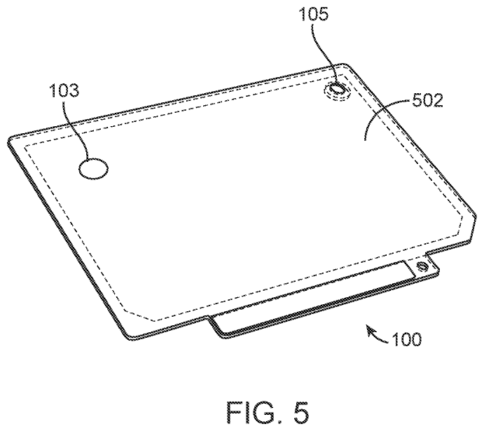

Referring to FIG. 5, microplate cover 100 can comprise one or more sensors. A sensor 103 of microplate cover 100 can comprise a pressure sensor (e.g., a gas pressure sensor), a pH sensor, a temperature sensor, a humidity or moisture sensor, or a gas content sensor (e.g., for measuring partial pressures of gases within the controlled environment). In some cases, microplate cover 100 can comprise a plurality of sensors distributed across the microplate cover to measure environmental conditions in separate sub-regions of the controlled environment (e.g. environmentally isolated sub-regions) or to monitor mixing, diffusion, respiration, or differential spatial consumption of reagents during an assay. In some cases, measurements made using sensor 103 can be used to determine experimental parameters such as sample respiration, or oxygen content in an environmentally isolated culture region or culture sub-region. In some cases, sensor 103 can be a detector operable by a computer (e.g., via wireless communication).

Control of moisture or humidity inside of an environmentally isolated culture region or culture sub-region can be important for the function of a microplate cover 100 and/or a joined microplate and cover 120. For example, many samples, such as cellular samples, require a humidified environment for survival. A humidified environment can be achieved in a culture system (e.g., a joined microplate and cover 120) by introducing a gas with a desired amount of humidification. More commonly, humidification can be achieved in a culture system by heating at least a portion of the culture system, wherein the system also contains a liquid medium or wherein the system is supplied with a humidification reservoir. Increased levels of humidity within a culture system (e.g., an environmentally isolated or sealed culture region or sub-region) can cause or can increase condensation on a surface of a microplate cover 100 or microplate 110, and increased condensation can impair the optical properties of a microplate cover or microplate. Further, biological function(s) of a sample (e.g., respiration of a cellular sample, the rate of which can vary with sample genotype or sample phenotype) can increase humidity levels within a closed culture environment (e.g., a culture region 112 or culture sub-region 114 of a joined microplate and cover 120).

In some cases, a microplate cover 100 comprising a hydrophobic material (e.g., constructed from or coated with a hydrophobic material) can decrease or slow the rate of condensation on the microplate cover and can preserve the optical properties of the microplate cover in a high-humidity environment. In some cases, a microplate cover 100 or a system comprising microplate cover 100 can comprise one or more features configured to vent, remove, or control humidity levels within a culture region 112 or culture sub-region 114. For example, humidity levels within a culture region or culture sub-region of a culture system (e.g., joined microplate and cover 120) can be controlled by injecting a gas or gas mixture having a higher or lower vapor content than the gas or gas mixture within the culture region or culture sub-region (e.g., via a gas management system in fluidic communication with the culture region or culture sub-region). In some cases, humidity levels within a culture region or culture sub-region can be controlled by actively removing or passively venting a gas or gas mixture (e.g., via a gas management system in fluidic communication with the culture region or culture sub-region).

In some cases, injection, removal, or venting of a gas or gas mixture into or from a culture region or culture sub-region can be accomplished by establishing a path of fluidic communication between the culture region or culture sub-region and a source of a gas or gas mixture and/or a gas exhaust. A port 105 in a microplate cover can comprise a path of fluidic communication between a culture region or culture sub-region and a source of a gas or gas mixture and/or a gas exhaust. In some cases, a port 105 can comprise a path of fluidic communication between a culture region or culture sub-region and a source of a gas or gas mixture and/or a gas exhaust without compromising other culture conditions within the culture region or culture sub-region. For example, a port 105 can allow gas exchange or liquid exchange with a culture region or sub-region while also providing a barrier against contamination. In some cases, a port 105 can preserve environmental isolation of a culture region or culture sub-region, provide a means of controlling humidity and condensation within the culture region or culture sub-region, and/or provide a path through which reagents (e.g., culture media, samples, or experimental stimuli) can be provided to or removed from a culture region or a culture sub-region.

In some cases, an apparatus comprising a microplate cover 100 and a port 105 can also comprise a sensor 103 to measure one or more environmental parameter (e.g., humidity, temperature, pH, or gas content) inside of a culture region or culture sub-region. In some cases, an apparatus can be configured to detect (e.g., to measure or to determine) a value of one or more environmental parameter inside of a culture region 112 or culture sub-region 114 (e.g., via measurements made by sensor 103 or measurements made through filter 107, lens 109, or through an optically permissive portion of microplate cover 100) and, optionally, to activate a system (e.g., a gas management system) to modulate the environmental parameter. In some cases, apparatus can be configured to detect (e.g., to measure or to determine) a value of one or more environmental parameter inside of a culture region 112 or culture sub-region 114 (e.g., via measurements made by sensor 103 or measurements made through filter 107, lens 109, or through an optically permissive portion of microplate cover 100) and, optionally, to activate a system (e.g., an imaging system) to make an measurement (e.g., to image a sample in the culture region or the culture sub-region or to image a sample in a different culture region or culture sub-region).

Microplate cover 100 can comprise a port 105. Port 105 may be an aperture formed though the microplate cover 100 to allow controlled respiration of the otherwise enclosed environment of a covered microplate and microplate cover. For example, port 105 can allow addition or removal of a gas or liquid from a portion or the entirety of the controlled environment created by joining microplate cover 100 with microplate 110 without the need to unjoin the microplate cover and microplate, which could lead to contamination of a sample or experimental variation. Port 105 can be configured to interface with a manifold for extraction or introduction of gases or liquids. For example, Port 105 can be configured to interface with a gas injector, a syringe, a syringe pump, a computer-controlled gas or liquid handling system, or with an exhaust. In some cases, port 105 can comprise a vent (e.g., a rectified vent or bidirectional port). Port 105 can comprise a membrane material that is easily punctured (e.g., a self-sealing polymer, rubber, or epoxy). For example, additional holes or apertures may be cut or formed through portions of microplate cover 100 at port 105. Microplate covers comprising one or more port 105 having an easily punctured material can allow for sample collection without removal of the cover plate without disturbing other samples in other contained spaces within the same sealed microplate. The easily punctured membrane of port 105 can be self-resealing, such that after the device used to puncture the membranes is removed or withdrawn from the microplate cover, the puncture hole formed in the membrane reseals itself.

Port 105 can comprise a membrane capable of controlling retention or ventilation of moisture during an assay. In some case, port 105 can comprise a flexible material (e.g., a self-sealing material such as a self-sealing polymer or rubber) can be used to introduce or extract reagents (e.g., inject, inoculate, vent, or remove, one or more sample, gas, liquid, drug, etc.) into or out of the space between a joined microplate and cover 120 (e.g., into or out of one or more culture region or culture sub-region). Port 105 may comprise a valve or a one-way gas or liquid rectifier.

Although FIG. 5 depicts the microplate cover 100 with a single aperture (e.g., port 105), in some embodiments, the microplate cover 100 may have more than one aperture, such as, for example, 2, 5, 6, 10, 12, 24, 48, 96, 384, 1536, from 2 to 6, from 6 to 12, from 12 to 24, from 24 to 48, from 48 to 96, from 96 to 384, from 384 to 1536, or more than 1536 apertures. In some embodiments, one or more aperture(s) 105 in the microplate cover may aid in the integration of sensors, lenses, light pipes, thermal control features, electronics, etc. onto or within microplate covers.

One or more membranes, such as membrane 502 may be attached over or otherwise cover port 105 to control retention or ventilation of moisture while ventilating (or introducing) gases within the enclosed microplate environment. In some cases, membrane 502 can comprise a material allowing only directional (e.g., rectified) flow of one or more gases (e.g., into the controlled environment or out of the controlled environment). Membrane 502 can also comprise a material allowing bidirectional flow of one or more gases.

Microplate cover 100 can comprise a plurality of ports 105. In some cases, one or more ports of the plurality of ports are disposed at a position on the microplate cover corresponding to the position(s) of one or more samples in a microplate when the microplate cover is joined to the microplate. In some cases, a port 105 can be configured to exchange gas or liquid (e.g., introduce or remove a gas or liquid) in only a sub-region (e.g., one microtiter well) of the microplate to which the microplate cover is joined. In some cases, port 105 can be configured to exchange or distribute a gas or liquid across a plurality of sub-regions of a microplate or to the entirety of the controlled environment formed by joining microplate cover 100 to microplate 110. In some cases, a first port disposed in microplate cover 100 can be used as an input port. In some cases, a second port disposed in microplate cover 100 can be used as an exhaust or outflow port. In some cases, a single port can be used as an input port and an outflow/exhaust port.

Port 105 can comprise a channel from an exterior surface of microplate cover 100 to an interior surface of microplate cover 100. Port 105 can comprise a channel from an exterior surface of microplate cover 100 to an interior surface of microplate cover 100 into one or more specific culture region(s) or sub-region(s) of the controlled environment formed between the joined microplate cover 100 and microplate 110. In some cases, port 105 comprises a solid or flexible structure (e.g., a cylinder or channel comprising three or more sides) that extends from the plane of a surface of the microplate cover into the space between the microplate cover and a microplate to which it is joined. For example, port 105 can comprise a channel that extends from the outer surface of microplate cover 100 toward the inner surface of a of microplate 110 and can be used to introduce one or more reagents (e.g., one or more gas, liquid, or drug) to a sample, region, or sub-region within the controlled environment during an assay (e.g., a diffusion study or assays comprising time-delayed environmental modification to media, such as nutrient source changes). In some case, port 105 can be used to deposit (e.g., inoculate) a sample on or within a sample media (e.g., solid or liquid three-dimensional culture matrices and media or matrices coated on a surface of microplate 110) in a culture region or sub-region located within joined microplate cover 100 and microplate 110. For example, a cellular sample (e.g., one or more yeast, bacteria, mammalian, plant, or other cell(s)) can be deposited on or within a solid media (such as agar) without breaking the seal of the joined microplate and microplate cover (e.g., without compromising the controlled environment) by delivering the cellular sample through port 105 using a needle, pipette, or other sample delivery system.

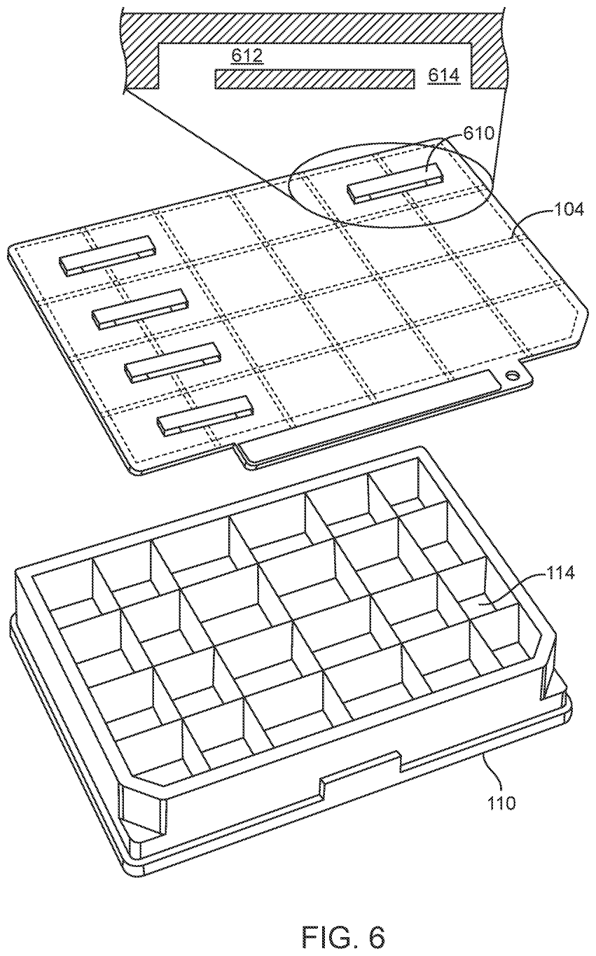

Referring now to FIG. 6, microplate cover 100 can comprise one or more channels 610. In some cases, a channel 610 of microplate cover 100 can be partially or fully disposed within microplate cover 100 and can facilitate introduction, extraction or distribution of one or more liquids, gases, or mixtures of gases. A channel 610 of microplate cover 100 can place in fluid communication two or more culture regions 112 or culture sub-regions 114 that are otherwise environmentally isolated from one another (e.g., by one or more dividers of microplate 110 that are sealed to microplate 100 by adhesive material 104. In some cases, two or more culture regions or culture sub-regions placed in fluid communication by channel 610 can allow the two or more culture regions or culture sub-regions to share environmental conditions such as pressure, temperature, gas composition, or humidity. In some cases, channel 610 can be in fluid communication with port 105. In some cases, channel 610 can comprise a port 105.

Channel 610 can comprise one or more portions parallel to, substantially parallel to, perpendicular to, or substantially perpendicular to the plane of microplate cover 100. Channel 610 can have one or more horizontal portion 612, which can have a horizontal axis in the plane of microplate cover 100. A horizontal portion 612 of channel 610 can be partially or fully disposed within the material of microplate cover 100. Channel 610 can have one or more vertical portion 614, which can have an axis perpendicular or substantially perpendicular to the plane of the largest surface of microplate cover 100. In some cases, channel 610 can comprise an aperture in a surface (e.g., an exterior surface or an interior surface) of microplate cover 100. Channel 610 can comprise a plurality of apertures in an interior surface of microplate cover 100. For example, channel 610 can comprise a plurality of apertures at a plurality of locations in one or more interior surface of microplate cover 100 corresponding to a plurality of environmentally isolated culture regions 112, culture sub-regions 114, or a combination of culture regions and culture sub-regions. In some cases, a channel 610 comprising an aperture open to an exterior surface of microplate cover 100 can place a culture region or culture sub-region within the space between microplate cover 100 and a microplate 110 joined to microplate 100 (e.g., a culture region or sub-region with which channel 610 is in fluid communication) in fluid communication with environmental conditions (e.g., pressure, humidity, temperature, gas mixtures, etc.) present exterior to the joined microplate 110 and microplate cover 100.

In some cases, channel 610 can comprise a selectable means of blocking fluid or gas flow through channel 610. For example, channel 610 can comprise a valve, a slidably connected door, or a hingedly connected door capable of blocking flow of a fluid or gas through channel 610. A selectable means of blocking fluid or gas flow through channel 610 can be located in a vertical portion 614 of channel 610, a horizontal portion 612 of channel 610, at an aperture of channel 610 open to an interior surface of microplate cover 100 or at an aperture of channel 610 open to an exterior surface of microplate cover 100.

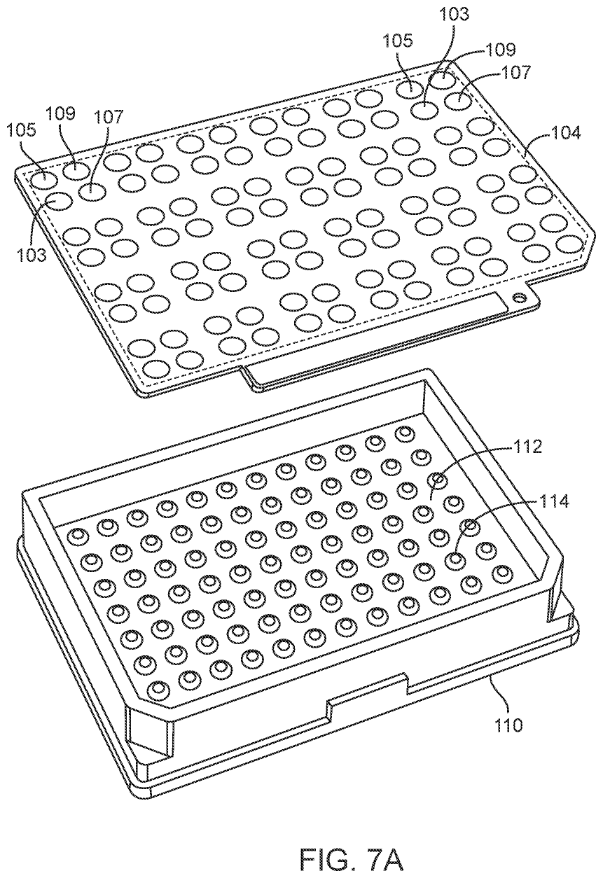

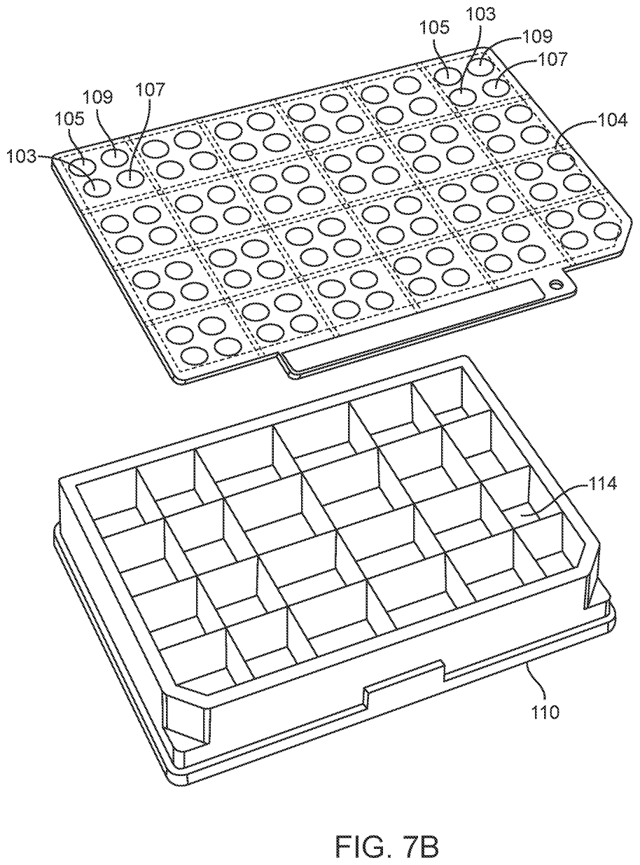

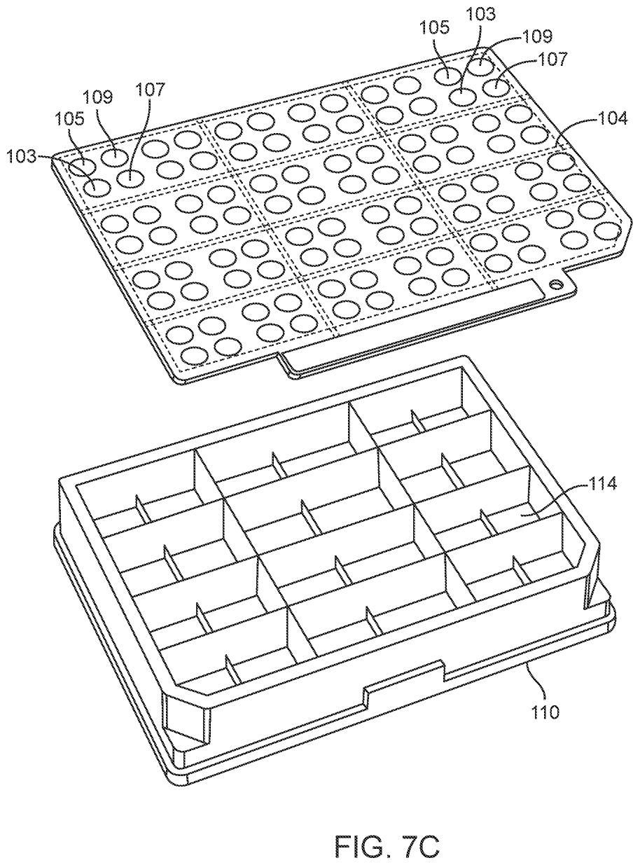

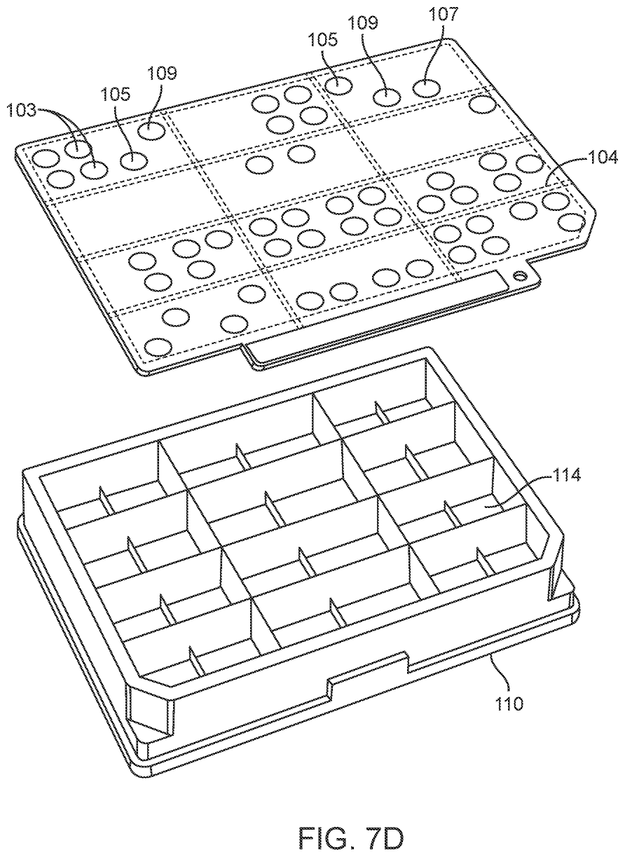

Referring now to FIG. 7A, a plurality of features of microplate cover 100 (e.g., sensor 103, port 105, filter 107, and/or lens 109) can be located on microplate cover 100 in patterns corresponding to positions of samples, culture regions, or culture sub-regions located in a space between joined microplate cover 100 and microplate 110. In some embodiments, a plurality of features of microplate 100 is grouped to allow multiple measurements and/or manipulations of similar samples (e.g., experimental repeats). For example, four similar or identical samples can be located in close proximity to one another in a culture region between microplate cover 100 and microplate 110, and sensor 103, port 105, filter 107, and lens 109 can be located in positions on microplate cover 100 above each of the samples. By positioning different features of microplate cover 100 over each of a plurality of similar or identical samples, it is possible to measure, image, and extract material from certain samples of the plurality of similar or identical samples without disturbing the other samples. In some cases, each of a plurality of a single type of feature of microplate cover 100 (e.g., sensor 103, port 105, filter 107, or lens 109) can be located in a position on microplate cover 100 that allows imaging, sensing, or manipulation of a different sample in a single culture region or culture sub-region.

Referring now to FIG. 7B and FIG. 7C, a microplate cover 100 can comprise a plurality of a single type of feature of microplate cover 100 (e.g., sensor 103, port 105, filter 107, or lens 109), wherein two or more of the features of the plurality of features are located positions on microplate cover 100 that allows imaging sensing, or manipulation of a different sample in a different culture region or culture sub-region 114. In some cases, each portion of microplate cover 100 encircled by adhesive material 104 comprises 1, 2, 3, 4, 5, 6, 7, 8, 9, 10, 11, 12, 12 to 24, 24 to 48, 48 to 96, 96 to 384, or 384 to 1536 features of microplate cover 100 (e.g., sensor 103, port 105, filter 107, and/or lens 109). In some cases, each portion of microplate cover 100 encircled by adhesive material 104 comprises no more than 1, 2, 3, 4, 5, 6, 7, 8, 9, 10, 11, 12, 12 to 24, 24 to 48, 48 to 96, 96 to 384, or 384 to 1536 of a single type of feature of microplate cover 100 (e.g., sensor 103, port 105, filter 107, or lens 109).

Referring now to FIG. 7D, a portion of microplate cover 100 encircled by adhesive material can comprise 0, 1, 2, 3, 4, 5, 6, 7, or 8 features of microplate cover 100 (e.g., sensor 103, port 105, filter 107, and/or lens 109). In some cases, a portion of microplate cover 100 encircled by adhesive material can comprise 8 to 12, 12 to 24, 24 to 48, 48 to 96, 96 to 384, or 384 to 1536 features of microplate cover 100 (e.g., sensor 103, port 105, filter 107, and/or lens 109). Features of microplate cover 100 (e.g., sensor 103, port 105, filter 107, and/or lens 109) can be arranged in any fashion on microplate cover. For example, features of microplate cover 100 can be arranged in regular or staggered rows and columns. Features of microplate cover 100 can also be arranged in rings or clusters of any number of features. In some cases, the position of one or more features of microplate cover 100 can correspond to the position of one or more samples within the joined microplate cover 100 and microplate 110.

Referring to FIGS. 9A and 9B, embodiments of microplate covers 100 with one or more layers or features attached to top or bottom surfaces of the respective microplate covers 100 are shown. The layers or features may include light diffusion features that diffuse focused light (from one or more external light sources) 910 to reduce glare on the samples within the microplate that are being imaged by an imaging system. These added layers may be round or otherwise shaped to match light source patterns and may have a clear center aperture for viewing by users or imaging systems, and may include optical filters to limit the wavelength of light illuminating the photographed subject area. In some embodiments, the layers or features may be arranged similarly to add or include light polarization, light focusing (via Fresnel or other lens types, etc), as shown.

An apparatus, as described herein, can further comprise a means of interfacing with one or more port(s) 105 or channel(s) 610 of a microplate cover 100. For example, an apparatus can comprise a gasket and/or tubing configured to join with port(s) 105 or channel(s) 610. A computer of an apparatus can control the means of interfacing with one or more ports to inject (e.g., add) or extract (remove) one or more gases or gas mixtures into the controlled environment.

Referring now to FIG. 9A, filter feature 107 can be an annularly shaped feature that may be a filter that modulates (e.g., reduces through attenuation, absorption, or reflection) light transmission of one or more wavelengths of light (such as notch or band-rejection filters) or preferentially allows certain wavelengths of light (such as a band-pass filter) or a diffuser that scatters light to provides more uniform illumination of the sample as compared to the same illumination without the diffuser.

Referring to FIG. 9B, a microplate can comprise lens feature 109 (e.g., a Fresnel lens feature). Lens feature 109 can alter the angle of incidence of light on the sample as compared to the incidence of light on the sample without the lens feature 109. In some cases, lens feature 109 can allow an imaging system with limited lensing capabilities (e.g., low power objective(s)) to image a sample with higher power through combined magnification of the imaging system's lens(es) and the lens feature. Apparatuses comprising a microplate cover having one or more lens feature 109 can increase the efficiency of an assay comprising step(s) in which samples are imaged at different magnification because, in some cases, an imaging objective can be translated laterally across between a transparent portion of microplate cover 100 and a portion of the microplate cover comprising lens feature 109 (e.g., wherein the transparent portion and the lens feature each allow the imaging system to image a samples under the same experimental conditions) more quickly than an imaging system can change objectives and refocus on a single sample.

In some cases, a lens feature 109 can comprise a light pipe or segment of optically conductive material, such as an optical fiber. In some cases, a lens feature 109 comprising a light pipe can guide a ray or beam of light to a specific culture region or culture sub-region (e.g., to illuminate a sample). In some cases, a lens feature 109 comprising a light pipe can be used to alter the angle of incidence at which a ray or beam of light strikes a sample. For example, the path of a ray or beam of light can be altered by a lens feature 109 comprising a light pipe in order to change the illumination provided to a sample (e.g., for better discrimination of three-dimensional features of the sample, such as colony height or morphology). In some cases, a lens feature 109 can comprise a light source, such as a light-emitting diode (LED). For example, a microplate cover 100 comprising a lens feature 109 which comprises an LED can provide specific directionality and intensity of illumination useful for imaging a sample, such as the morphology, height, or three-dimensional features of a sample.

In addition to a microplate cover, as described herein, an apparatus can comprise a computer comprising a processor and a non-transitory memory with instructions thereon executable by the processor to perform steps of a method. For example, an apparatus can comprise a computer configured to operate any or all of a liquid handling system, one or more independent gas management system, one or more independent tray or microplate handling system (e.g., a tray or microplate handling system comprising an alignment pin), one or more light sources 910, one or more heating element, one or more imaging system 912, one or more detector of an imaging system 912, or software for sample tracking or data analysis. In some cases, an apparatus, as described herein comprises one or more light sources 910, a heating element, an imaging system 912, one or more detector (e.g., of imaging system 912), or software for sample tracking or data analysis.

An apparatus described herein is also capable of executing the steps of an assay, such as a biological assay. For example, an apparatus comprising a computer, a microplate 110, a microplate cover 100, and a sample can be configured to produce an image or a plurality of images (e.g., a time-lapse series of images or a Z-stack image at a given time point) of a plurality of samples.

In some cases, an apparatus comprising a computer, a microplate 110, a microplate cover 100, and a sample can be configured to perform one or more step of an assay at certain times during the assay. For example, an apparatus as described herein can be configured to perform a step of an assay, such as imaging a sample, extracting a sample, or adding a treatment to a sample located in a culture region or sub-region (e.g., from a colony of cells located on or within a solid culture matrix), at pre-defined time points supplied to the computer by a user or by a program stored on the computer. In some cases, an apparatus as described herein can be configured to perform one or more step of an assay, such as imaging a sample, extracting a sample, or adding a treatment to a sample located in a culture region or sub-region as a result of a conditional event, such as a measurement made by a sensor 103. For example, an apparatus can be configured to image or extract a sample after a sensor 103 has measured that a threshold value has been exceeded.

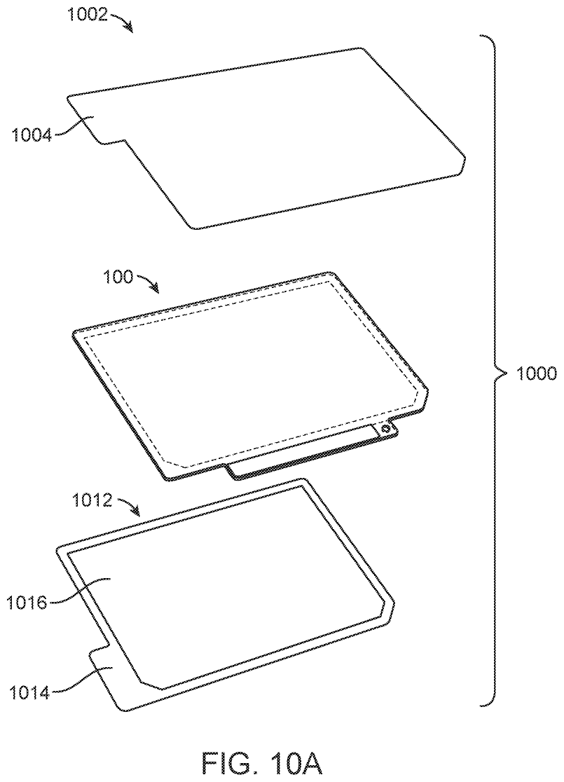

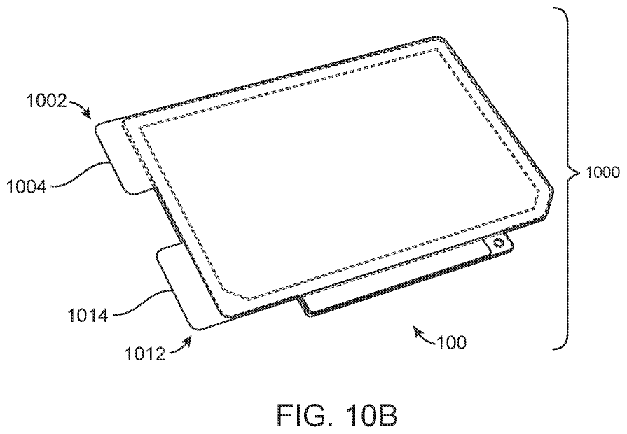

Referring to FIGS. 10A and 10B, an embodiment of a microplate cover assembly 1000, which can be useful as part of a kit for assaying a sample, is shown. In some embodiments, the microplate cover assembly 1000 may include a microplate cover 100 and protective covers or liners 1002, 1012 on surfaces to protect, facilitate assembly of, and/or enable application of coatings upon the microplate cover. For example, in the exploded view shown in FIG. 3A, a microplate cover assembly 1000 may include an upper removable liner 1002 that covers and protects the upper surface of the microplate cover 100 (e.g., to preserve optical clarity, to prevent scratches and smudges, or to preserve sterility and prevent contamination) and a lower removable liner 1012 that covers and protects the lower surface of the microplate cover 100. The lower surface of the microplate cover 100 may be the microplate facing surface, for example, the surface of the microplate cover that faces the microplate when the microplate cover 100 is installed on a microplate. The upper surface may be a surface opposite the microplate facing surface, such as an outer surface that faces away from the microplate when the microplate cover 100 is installed on a microplate.

The removable liners 1002, 1012 may have light-tack, low-residue adhesive applied to keep the liners attached to the microplate cover 100 until removed by users. Pull tabs 1004, 1012 may extend from the liners to enable easy removal without excessive handling by users, and to ensure alignment of the microplate cover with fixtures, packaging, or other equipment.

One or more removable liners 1002, 1012 may also have an open inner section to cover adhesive strips or delicate surfaces, and to enable manual or automated coating of selected surfaces of the microplate cover (e.g. with anti-condensation coatings such as hydrophobic materials, anti-glare coatings, etc.) without contaminating or damaging adhesive strips or sensitive surfaces of the microplate cover. For example, removable liner 1012 includes an open area 1016 that extends between the microplate facing surface and the outer surface of the removable liner 1012.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-72. (canceled)

73. An apparatus for assaying a sample, comprising:
a microplate cover comprising:
an interface configured to join the microplate cover and a microplate to form a plurality of culture sub-regions,
a first microplate cover sub-region comprising a first plurality of microplate cover features, wherein the first plurality of microplate cover features is located on the microplate cover in a position corresponding to a first culture sub-region, and
a second microplate cover sub-region comprising a second plurality of microplate cover features, wherein the second plurality of microplate cover features is located on the microplate cover in a position corresponding to a second culture sub-region.

74. The apparatus of claim 73, wherein the first culture sub-region of the plurality of culture sub-regions is substantially environmentally isolated from the second culture sub-region of the plurality of culture sub-regions.

75. The apparatus of claim 73, wherein the first microplate cover feature of the first plurality of microplate cover features is selected from a port, a sensor, a lens, and an optical filter.

76. The apparatus of claim 75, wherein the first microplate cover feature of the first plurality of microplate cover features is an optical filter.

77. The apparatus of claim 75, wherein a second microplate cover feature of the first plurality of microplate cover features is selected from a port, a sensor, a lens, and an optical filter.

78. The apparatus of claim 75, wherein a first microplate cover feature of the second plurality of microplate cover features is selected from a port, a sensor, a lens, and an optical filter.

79. The apparatus of claim 78, wherein a second microplate cover feature of the second plurality of microplate cover features is selected from a port, a sensor, a lens, and an optical filter.

80. A method for assaying a sample, comprising:
joining a microplate cover comprising an orientation feature to a microplate to define a plurality of culture sub-regions;
positioning the microplate based on the orientation feature; and
imaging a first aliquot of a sample located in a first culture sub-region of the plurality of culture sub-regions.

81. The method of claim 80, wherein the imaging comprises measuring a three-dimensional feature of the first aliquot.

82. The method of claim 81, wherein the first aliquot comprises a three-dimensional colony of adherent cells.

83. The method of claim 80, wherein imaging the first aliquot of the sample comprises producing a Z-stack image.

84. The method of claim 80, wherein positioning the microplate comprises positioning the first aliquot in a first orientation relative to a light source based on the orientation feature at a first experimental time point before imaging the first aliquot.

85. The method of claim 84, further comprising re-positioning the microplate in the first orientation relative to the light source based on the orientation feature at a second experimental time point before repeating the imaging of the first aliquot.

86. The method of claim 80, wherein the first culture sub-region is substantially environmentally isolated from a second culture sub-region of the plurality of culture sub-regions, the second culture sub-region comprising a second aliquot of the sample.

87. An apparatus for assaying a sample, comprising:
a microplate cover comprising:
a plurality of optical filters, and
an interface configured to join the microplate cover and a microplate.

88. The apparatus of claim 87, wherein a first optical filter of the plurality of optical filters comprises different optical properties than a second optical filter of the plurality of optical filters.

89. The apparatus of claim 87, wherein a first optical filter of the plurality of optical filters polarizes light.

90. The apparatus of claim 87, wherein the first optical filter is a bandpass filter.

91. The apparatus of claim 87, wherein the microplate cover further comprises a feature selected from a sensor, a port, or a lens.

92. The apparatus of claim 91, wherein the feature is a lens.
